(19) United States
(12) Patent Application Publication
(10) Pub. No.: US 2021/0030649 A1
(43) Pub. Date: Feb. 4, 2021

(54) COSMETIC COMPOSITION FOR WET SKIN

(71) Applicant: BEIERSDORF AG, Hamburg (DE)

(72) Inventors: Osvaldo Jose Fabian CRUZ CERVANTES, Leon (MX); Luis Arturo CARBAJAL AVILA, Hamburg (DE)

(21) Appl. No.: 17/043,037

(22) PCT Filed: Apr. 5, 2018
(86) PCT No.: PCT/EP2018/058658

§ 371 Date: Sep. 29, 2020

(51) Int. Cl. A61K 8/37; A61K 8/34; A61K 8/891; A61Q 17/04

(52) U.S. Cl. CPC A61K 8/37; A61K 8/347; A61K 2800/594; A61Q 17/04; A61K 8/891

(57) ABSTRACT

The present invention belongs to the cosmetic field and relates to a cosmetic composition containing a specific combination of an ester, a polymer and water, which allows to reduce the white appearance when applied to wet skin.

The present invention belongs to the cosmetic field and relates to a cosmetic composition containing a specific combination of an ester, a polymer and water, which allows to reduce the white appearance when applied to wet skin.

A beautiful and attractive appearance is a desire for many people. One typical sign of such an appearance is a healthy and smooth looking skin. Therefore, in order to take care on the skin, it is for many people a daily routine to apply cosmetic products such as body lotions and sunscreen compositions.

For example, compositions containing UV-filters are commonly used to protect the skin from the ultraviolet radiation of the sunlight. In addition to the acute damage (sunburn), long-term damage such as an increased risk of skin cancer occurs in case of excessive irradiation with light from the UVB range (wavelength: 280-320 nm). The excessive exposure of UVB and UVA radiation (wavelength: 320-400 nm) also leads to a weakening of the elastic and collagen fibers of the connective tissue. This leads to numerous photo-toxic and photo-allergic reactions and results in premature skin ageing.

To protect the skin, a number of light protection filter substances have been developed, which can be used in cosmetic preparations. These UVA and UVB filters are grouped in the most developed countries in the form of positive lists such as Annex VI of the Regulation (EC) No 1223/2009 of the European Parliament and of the Council.

However, the wide variety of commercially available sunscreens must not obscure the fact that these preparations of the prior art have a number of disadvantages.

For example, customers often complain about the fact that conventional sunscreen formulations, meaning those which comprise at least 40% of water and a lipid phase, produce a strong white appearance when applied on wet skin. Furthermore, once applied to wet skin it often takes a while until the white appearance disappears.

The document DE 102013203045 A1 discloses anhydrous sunscreen composition and addresses the issue of white residues of particulate filter substances after application of sunscreens. However, there is no discloser of a cosmetic compositions according to the present invention.

Furthermore, the person skilled in art is aware of the document US 20150352035 A1, which discloses sunscreens which appear to be clear when applied to the skin. Again, there is no disclosure contained which can direct the person skilled in art to the present invention.

The objective of the present invention is therefore to provide a cosmetic composition which does not suffer the disadvantages of the state of the art. In particular it was one objective of the present invention to provide a cosmetic composition which reduces the white appearance after application to wet skin. It was another objective of the present invention to provide a cosmetic composition whose white appearance quickly disappears after application to wet skin. Thereby, it is further desirable that such a composition preferably does not suffer stability incompatibilities. It is further desirably that such a composition has a fresh sensation after application to the skin.

It was surprisingly found that the objectives described above can be solved by the present invention.

The invention is the use of a combination of
a) at least one ester having a dielectric constant of at least 4.0 at a temperature of 25° C.; and
b) at least one acrylates/C10-30 alkyl acrylate crosspolymer, which is defined by the fact that, if the polymer is dissolved in a total quantity of 0.2% by weight in water and the resulting mixture is adjusted to a pH of 7.5, the resulting mixture has a viscosity in the range from 4500 mPa·s to 5500 mPa·s, whereby the viscosity is measured using a Brookfield RV viscometer, spindle RV 4, 20 rpm without Helipath, at 20° C. ambient temperature and 20° C. sample temperature;

in a cosmetic composition containing, calculated to the total weight of the composition, at least 40% by weight of water to reduce the white appearance of the composition after application to wet skin and/or to reduce the time until the white appearance of the composition disappears after the application to wet skin.

The term “after application” refers herein to a period starting with the application and ending preferably 5 minutes, more preferably 2 minutes and most preferably 1 minute after finishing the application of the composition.

A further object of the present invention is a cosmetic composition containing, calculated to the total weight of the composition,
a) at least one ester having a dielectric constant of at least 4.0 at a temperature of 25° C.;
b) at least one acrylates/C10-30 alkyl acrylate crosspolymer, which is defined by the fact that, if the polymer is dissolved in a total quantity of 0.2% by weight in water and the resulting mixture is adjusted to a pH of 7.5, the resulting mixture has a viscosity in the range from 4500 mPa·s to 5500 mPa·s, whereby the viscosity is measured using a Brookfield RV viscometer, spindle RV 4, 20 rpm without Helipath, at 20° C. ambient temperature and 20° C. sample temperature; and
c) at least 40% by weight of water.

According to the invention the pH-adjustment for the measurement of the viscosity of the acrylates/C10-30 alkyl acrylate crosspolymer is either done by the addition of sodium hydroxide or citric acid.

All the weight percentages (% by weight) given below relate, unless otherwise stated, to the total weight of the cosmetic composition. If ratios of certain components are disclosed in the following description, these ratios refer, unless otherwise stated, to weight ratios of the components.

Unless otherwise stated, all tests and measurements were performed under “normal conditions”. The term “normal conditions” refers to 20° C., 1013 hPa and a relative humidity of 50%.

All disclosed values for the dielectric constant relate to a sample temperature of 25° C. Furthermore, the disclosed values for the dielectric constant relate measurements done at 1013 hPa and a relative humidity of 50%.

For the purposes of the present disclosure, the term “free from” means that the proportion of the respective substance is less than 0.05% by weight. This ensures that entrainments or impurities with these substances are not included as “free from” according to the invention.

The term “skin” refers solely to human skin.

In the following description the terms “according to the invention”, “preferred according to the invention” and so on are always directed to the use according to the invention and to the cosmetic composition according to the invention.

According to the present invention the cosmetic composition contains at least one ester having a dielectric constant of at least 4.0 at a temperature of 25° C.

The ester a) is preferably not an organic UV-filter.

Furthermore, it is preferred if the dielectric constant of the ester a) is at least 4.5, more preferably 5.0 and most preferably 5.2, wherein all values for the dielectric constant related to a temperature of 25° C.

Moreover, it is preferred if the dielectric constant of the ester a) is less than 8.0, more preferred less than 7.0 and most preferred less than 6.0, wherein all values for the dielectric constant related to a temperature of 25° C.

Following the limitation of the lower and upper limit of the dielectric constants for the ester, it is also advantageous, if the ester a) has a dielectric constant in the range from 4.5 to 8.0, preferably 5.0 to 7.0 and more preferably 5.2 to 6.0, wherein all values for the dielectric constant related to a temperature of 25° C.

According to the present invention the dielectric constant of an ester can be determined using dielectric constant meter, e.g. BI-870 Dielectric Constant Meter from the Fa. Brookhaven Instruments Corporation, USA.

The following table indicates the dielectric constants of several organic substances including a number of ester. The dielectric constants are given for a sample temperature of 25° C.

TABLE 1

| INCI | Dielectric constant |
|---|---|
| Isodecyl Malate | 6.0 |
| Lauryl Lactate | 5.6 |
| C-12-15 Alkyl Lactate | 5.4 |
| Diisopropyl Adipate | 5.4 |
| Diisopropyl Sebacate | 4.7 |
| Diethylhexyl Adipate | 4.2 |
| Butyl Oleate | 4.0 |
| Caprylic/Capric Triglycerides | 3.8 |
| C12-15 Alkyl Benzoate | 3.8 |
| Isopropyl Myristate | 3.2 |
| Ethylhexyl Palmitate | 3.1 |
| Ethylhexyl Stearate | 3.1 |
| Isocetyl Stearate | 3.0 |
| Octyldodecyl Stearate | 2.8 |

According to the present invention it is preferred if the ester a) having a dielectric constant of at least 4.5 at a temperature of 25° C. is chosen from the group of diisopropyl adipate, lauryl lactate, dibutyl adipate, diisopropyl sebacate, diethylhexyl adipate, isodecyl malate and C12-15 alkyl lactate whereby diisopropyl adipate is chosen as the most preferred ester.

Furthermore, it is also preferred if the total quantity of the at least one ester a) having a dielectric constant of at least 4.5 at a temperature of 25° C., which is preferably not an organic UV-filter, is in the range from 1% to 12% by weight, more preferably 1.5% to 6% by weight and most preferably 2% to 5% by weight, calculated to the total weight of the cosmetic composition.

Furthermore, in one embodiment of the invention it is also preferred if the total quantity of the at least one ester a) having a dielectric constant of at least 4.5 at a temperature of 25° C., which is chosen from the group of diisopropyl adipate, dibutyl adipate, lauryl lactate, diisopropyl sebacate, isodecyl malate and C12-15 alkyl lactate, is in the range from 1% to 12% by weight, more preferably 1.5% to 6% by weight and most preferably 2% to 5% by weight, calculated to the total weight of the cosmetic composition.

Still, in another embodiment of the invention it is also preferred if the total quantity of the at least one ester a) having a dielectric constant of at least 4.5 to 8.0 at a temperature of 25° C. is in the range from 1% to 12% by weight, more preferably 1.5% to 6% by weight and most preferably 2% to 5% by weight, calculated to the total weight of the cosmetic composition.

In another embodiment of the invention it is also preferred if the total quantity of the at least one ester a) having a dielectric constant of at least 5.0 to 7.0 at a temperature of 25° C. is in the range from 1% to 12% by weight, more preferably 1.5% to 6% by weight and most preferably 2% to 5% by weight, calculated to the total weight of the cosmetic composition.

Moreover, in another embodiment of the invention it is also preferred if the total quantity of the at least one ester a) having a dielectric constant of at least 5.2 to 6.0 at a temperature of 25° C. is in the range from 1% to 12% by weight, more preferably 1.5% to 6% by weight and most preferably 2% to 5% by weight, calculated to the total weight of the cosmetic composition.

In a preferred embodiment of the invention diisopropyl adipate is contained as ester a) having a dielectric constant of at least 4.5 at a temperature of 25 ° C. and the total quantity of diisopropyl adipate is in the range from 1% to 12% by weight, more preferably 1.5% to 6% by weight and most preferably 2% to 5% by weight, calculated to the total weight of the cosmetic composition.

An acrylates/C10-30 alkyl acrylate crosspolymer which is according to the present invention is commercially available under the trade name Pemulen™ EZ-4U sold by the Fa Lubrizol.

Viscosity measurements which were made according to invention indicated a viscosity of 4900 mPa·s for Pemulen™ EZ-4U.

Preferred compositions according to the invention are further therein characterized that the total quantity of acrylates/C10-30 alkyl acrylate crosspolymers, which are defined by the fact that, if the polymer is dissolved in a total quantity of 0.2% by weight in water and the resulting mixture is adjusted to a pH of 7.5, the resulting mixture has a viscosity of less than 4000 mPa·s and/or more than 6000 mPa·s, whereby the viscosity is measured using a Brookfield RV viscometer, spindle RV 4, 20 rpm without Helipath, at 20° C. ambient temperature and 20° C. sample temperature, is less than 0.05% by weight and more preferably 0% by weight, calculated to the total weight of the cosmetic composition.

Furthermore, in terms of the present invention it is preferred if the at least one acrylates/C10-30 alkyl acrylate crosspolymer b) is contained in cosmetic composition in a total quantity in the range from 0.15% to 0.55% by weight , more preferably 0.18% to 0.5% by weight and most preferably 0.2% to 0.4% by weight, calculated to the total weight of the composition.

Additionally, preferred cosmetic compositions according to the invention contain water in a total quantity from 40% by weight to 90% by weight, more preferably from 45% by weight to 80% by weight and most preferably from 50% weight to 70% by weight, calculated to the total weight of the composition.

According the present invention it is further preferred, if the total quantity of all non-silicone oils, which have a dielectric constant of less than 4.0 at 25° C. and which are not an organic UV-filter, is less than 1% by weight, more preferably less than 0.8% by weight, more preferably less than 0.5% by weight, more preferably less than 0.2% by weight and most preferably less than 0.01% by weight, calculated to the total weight of the composition.

It is further especially preferred, if the total quantity of C12-15 alkyl benzoate, mineral oil, caprylic/capric triglyceride and/or ethylhexyl palmitate in the cosmetic composition is less than 0.2% by weight and more preferably 0% by weight, calculated to the total weight of the composition.

Specific cosmetic compositions of the present invention are preferably characterized in that they contain silicone oils in a total quantity of 0.1% by weight to 1% by weight, calculated to the total weight of the composition. Thereby, it is even more preferred if the silicone oil is dimethicone. Preferred dimethicones are further therein characterized that they have a viscosity of 50 to 200 cSt at 25° C.

Preferred cosmetic compositions according to the present invention may further comprise solid particles which are water insoluble and are not a liquid or gas at any temperature below 95° C. at 1013 hPa. A preferred example of such a particle is silica. In the case the cosmetic composition according to the invention contains solid particles, in particular silica, it is further preferred if the total quantity of the particles is in the range from 0.3% by weight to 2% by weight, calculated to the total weight of the composition.

Generally, it is further preferred if the total quantity of any further polymeric rheology modifier, selected from the group of carbomers, sodium polyacrylate and/or further polymers which are polymerized from a mixture containing acrylic acid, methacrylic acid, vinylpyrrolidone and/or vinyl acetate, is less than 0.2% by weight, more preferably less than 0.1% by weight and most preferably 0% by weight, calculated to the total weight of the composition. That means it most preferred if the cosmetic composition does not contain a rheology modifier selected from the group of carbomers, sodium polyacrylate and/or further polymers which are polymerized from a mixture containing acrylic acid, methacrylic acid, vinylpyrrolidone and/or vinyl acetate.

Moreover, it is preferred according to the present invention if the cosmetic composition contains polymers which are polymerized from a mixture containing polyols and di- or poly-isocyanate in a total quantity of 0% by weight, calculated to the total weight of the composition.

Furthermore, preferred embodiments of the present invention are therein characterized that the composition contains at least one organic UV-filter.

According to the invention organic UV-filters are those organic compounds which are known from the list of UV filters allowed in cosmetic products from Annex VI of the Regulation (EC) No 1223/2009 of the European Parliament and of the Council, including all amendments and corrections made until the 1^{st} of November 2017. The Regulation (EC) No 1223/2009 of the European Parliament and of the Council, the amendments and corrections can be accessed via the website http://eur-lex.europa.eu/homepage.html.

Organic UV-filter which are preferred according to the invention are selected from the group of compounds comprising camphor benzalkonium methosulfate, homosalate, benzophenone-3, phenylbenzimidazole sulfonic acid, terephthalylidene dicamphor sulfonic Acid, butyl methoxydibenzoylmethane, benzylidene camphor sulfonic acid, octocrylene, polyacrylamidomethyl benzylidene camphor, PEG-PABA, isoamyl P-methoxycinnamate, ethylhexyl triazone, drometrizole trisiloxane, diethylhexyl butamido triazone, 4-methylbenzylidene camphor, ethylhexyl salicylate, ethylhexyl dimethyl PABA, benzohenone-4, benzophenone-5, methylene bis-benzotriazolyl tetramethylbutylphenol, disodium phenyl dibenzimidazole tetrasulfonate, bis-ethylhexyloxyphenol methoxyphenyl triazine, polysilicone-15, diethylamino hydroxybenzoyl hexyl benzoate and tris-biphenyl triazine. Organic UV-filters which are more preferred are selected from the group of compounds comprising homosalate, phenylbenzimidazole sulfonic acid, butyl methoxydibenzoylmethane, octocrylene, ethylhexyl triazone, ethylhexyl salicylate, benzohenone-4, bis-ethylhexyloxyphenol methoxyphenyl triazine and polysilicone-15.

The organic UV-filters which are most preferred according to the invention are selected from the group bis-ethylhexyloxyphenol methoxyphenyl triazine, homosalate, ethylhexyl salicylate, octocrylene and butyl methoxydibenzoylmethane.

According to the invention it is preferred if the total quantity of the organic UV-filters contained in the cosmetic composition is in the range from 1% by weight to 35% by weight, more preferably 8% by weight to 30% by weight and most preferably 10% by weight to 28% by weight, calculated to the total weight of the composition.

In a preferred case of the invention the cosmetic composition contains at least one organic UV-filter selected from the group of compounds comprising bis-ethylhexyloxyphenol methoxyphenyl triazine, homosalate, ethylhexyl salicylate, octocrylene and butyl methoxydibenzoylmethane, whereby the total quantity of the organic UV-filters selected from the group of compounds comprising bis-ethylhexyloxyphenol methoxyphenyl triazine, homosalate, ethylhexyl salicylate, octocrylene and butyl methoxydibenzoylmethane is in the range from 1% by weight to 35% by weight, more preferably 8% by weight to 30% by weight and most preferably 10% by weight to 28% by weight, calculated to the total weight of the composition.

In the case that the cosmetic composition of the present invention contains bis-ethylhexyloxyphenol methoxyphenyl triazine it is preferred if the total quantity of bis-ethylhexyloxyphenol methoxyphenyl triazine is in the range from 0.1% by weight to 5% by weight, more preferably from 0.5% by weight to 4.8% by weight and most preferably from 1% by weight to 4.5% by weight, calculated to the total weight of the composition.

In the case that the cosmetic composition of the present invention contains homosalate it is preferred if the total quantity of homosalate is in the range from 1% by weight to 15% by weight, more preferably from 3% by weight to 10% by weight and most preferably from 5% by weight to 8% by weight, calculated to the total weight of the composition.

In the case that the cosmetic composition of the present invention contains ethylhexyl salicylate it is preferred if the total quantity of ethylhexyl salicylate is in the range from 1% by weight to 12% by weight, more preferably from 2% by weight to 8% by weight and most preferably from 3% by weight to 6% by weight, calculated to the total weight of the composition.

In the case that the cosmetic composition of the present invention contains octocrylene it is preferred if the total quantity of octocrylene is in the range from 0.1% by weight to 10% by weight, more preferably from 1% by weight to 9% by weight and most preferably from 2% by weight to 8% by weight, calculated to the total weight of the composition.

In the case that the cosmetic composition of the present invention contains butyl methoxydibenzoylmethane it is preferred if the total quantity of butyl methoxydibenzoylmethane is in the range from 0.5% by weight to 10% by weight, more preferably from 1% by weight to 8% by weight and most preferably from 3% by weight to 5% by weight, calculated to the total weight of the composition.

Furthermore, preferred compositions according to the invention are therein characterized that the compositions do not contain ethylhexyl methoxycinnamate.

Moreover, the cosmetic compositions of the present invention are preferably free from inorganic UV-filter, especially free from zinc oxide and titanium dioxide. It is especially preferred, if zinc oxide and titanium dioxide are not contained in the cosmetic composition of the present invention.

In addition the cosmetic composition according to the present invention may advantageously contain tapioca starch. In the case the cosmetic composition contains tapioca starch, the total quantity of tapioca starch is preferably in the range from 0.5% to 3% by weight, calculated to the total weight of the composition.

In some cases, preferred cosmetic compositions according to the present invention are characterized in that the compositions contain xanthan gum. In the case that a cosmetic composition according to the invention contains xanthan gum, the total quantity of xanthan gum is preferably in the range from 0.1% to 0.5% by weight, calculated to the total weight of the composition.

According to the invention, it is further preferred if the cosmetic composition contains phenoxyethanol. In the case the cosmetic composition contains phenoxyethanol the total quantity of phenoxyethanol is preferably in the range from 0.1% by weight to 2% by weight and more preferably from 0.4% by weight to 1% by weight, calculated to the total weight of the composition.

Moreover, preferred cosmetic compositions of the present invention are therein characterized that they contain ethylhexylglycerin, whereby it is further preferred if the total quantity of ethylhexylglycerin is in the range from 0.05% to 0.5% by weight, calculated to the total weight of the composition.

Furthermore, it is preferred if the cosmetic composition does not contain an alkylparabene such as methyl parabene and/or butyl parabene.

Further preferred cosmetic compositions according to the invention contain glycerol in a total quantity from 0.9% to 10% by weight, more preferably from 2% to 8% by weight calculated to the total weight of the composition.

In addition, preferred cosmetic compositions are characterized in that they contain ethanol. In the case that ethanol is contained in the compositions according to the invention, it if further preferred if the total quantity of Ethanol is in the range from 1% by weight to 10% by weight, more preferably from 2% by weight to 6% by weight, calculated to the total weight of the composition. Compositions which fulfill this criteria have the additional advantage that the compositions can be applied to sensitive skin whiteout causing a burning effect.

According to the invention the cosmetic composition may preferably contain at least one compound selected from the group of limonene, linalool, citral, alpha-isomethylionone and geraniol.

Embodiments of the present invention which are advantageous according to the invention are characterized in that the cosmetic composition contains as further ingredients one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, tocopheryl acetate, dihydroxyacetone, glycyrrhetinic acid, 8-hexadecene-1,16-dicarboxylic acid, glycerylglycose, (2-hydroxyethyl) urea and/or licochalcone A.

Furthermore, it was noted that preferred cosmetic compositions according to the invention are therein characterized that the cosmetic composition contains a gel structure, preferably a micro-gel structure.

The cosmetic compositions according to the invention are in preferred cases further therein characterized that the composition is an emulsion, more preferably a Hydrodispersion.

Hydrodispersions consist of a liquid lipid phase in an external aqueous phase, whereby the stability of the multiphase system is ensured in that the aqueous phase contains a gelling agent forming a gel structure, in which the lipid droplets are stably suspended. Therefore, in contrast to emulsions, hydrodispersions contain less than 2% by weight of emulsifiers or preferably no emulsifiers at all. The term “further emulsifier” is directed to the fact that in some instances the polymer acrylates/C10-30 alkyl acrylate crosspolymer is described as polymeric emulsifier. Accordingly, it is further preferred for the case that if the cosmetic composition is in form of a hydrodispersion that the total concentration of further emulsifiers is less than 2% by weight, more preferably less than 1% by weight and most preferably 0% by weight, calculated to the total weight of the composition.

Moreover, it is further preferred if the cosmetic composition according to the invention has a pH value in the range from 4.5 to 8.

Additionally, preferred cosmetic compositions of the present invention are therein characterized that they have a viscosity in the range from 1500 mPa·s to 5500 mPa·s, whereby the viscosity is measured using a Brookfield RV viscometer, spindle RV 4, 20 rpm without Helipath, at 20° C. ambient temperature and 20° C. sample temperature.

Generally, the cosmetic compositions according to the present invention can be prepared by common formulation techniques for emulsions. However, in the case the cosmetic composition according to the invention contains an UV-filter which is soluble in water it may happen that the water-soluble UV-filter affects the gel formation of the acrylates/C10-30 alkyl acrylate crosspolymer resulting in a loss of viscosity. It was surprisingly found that this problem can be addressed by adding the water-soluble UV-filter after the mixing of all other ingredients. This means the water-soluble UV-filter are added after the formation of the gel structure.

### EXAMPLES

The following examples should illustrate the compositions of this invention, without intending to limit the invention to these examples. The numerical values in the examples are percentages by weight, based on the total weight of the preparations.

The whitening effect of the cosmetic compositions was studied in the following manner.
1. A section of 80 cm² was marked on the forearm of a participant taking part in the study
2. 75 μl of water were spilled on the marked section
3. 25 μl of the cosmetic compositions was added to the marked section
4. With a finger 5 circles were drawn on the marked section to distributed the water and the cosmetic composition evenly
5. The whiteness of the composition was evaluated (1=none, 2=almost noticeable, 3=medium, 4=strong white)
6. The chronometer was started immediately after distributing the water and the cosmetic composition in the marked section
7. The time was recorded until the white marks disappeared

Each composition was investigated 12 times. The results of the study are presented in the table below. The example named Inv. X is a composition according to the invention, while Com X represents a comparative test, wherein X stands for an integer number.

| INCI | Inv. 1 | Inv. 2 | Com 1 |
|---|---|---|---|
| Diisopropyl Adipate | 3 | 5 | 0 |
| C12-15 Alkyl Benzoate | 0 | 0 | 3 |
| Dimethicone | 0.3 | 0.3 | 0.3 |
| Silica Dimethyl Silylate | 1 | 1 | 1 |
| Silica | 0.5 | 0.5 | 0.5 |
| Tapioca Starch | 0.85 | 0.85 | 0.85 |
| Glycerin | 0.9 | 0.9 | 0.9 |
| Methylpropanediol | 2 | 2 | 2 |
| Sodium Hydroxide | q.s. | q.s. | q.s. |
| Phenoxyethanol | 0.8 | 0.8 | 0.8 |
| Carbomer | 0 | 0.16 | 0 |
| Acrylates/C10-30 Alkyl Acrylate | 0.2 | 0.5 | 0.25 |
| Crosspolymer (Pemulen EZ4U, Lubrizol) | | | |
| Alcohol Denat. | 3.8 | 3.8 | 3.8 |
| Trisodium EDTA | q.s. | q.s. | q.s. |
| Homosalate | 8 | 8 | 8 |
| Octocrylene | 2 | 2 | 2 |
| Ethylhexyl Salicylate | 4.5 | 4.75 | 4.5 |
| Bis-Ethylhexyloxyphenol Methoxyphenyl | 1 | 3.5 | 1 |
| Triazine | | | |
| Butyl Methoxydibenzoylmethane | 3.5 | 4.75 | 3.5 |
| Ethylhexyl Triazone | 0 | 4 | 0 |
| Phenylbenzimidazole Sulfonic Acid | 0 | 2 | 0 |
| Polysilicone-15 | 0 | 0 | 0 |
| Aqua | ad 100 | ad 100 | ad 100 |
| Whiteness after application | 1.2 | 1.2 | 3 |
| Time until white residues disappeared | 2 | 3 | 83 |
| (Averaqe in seconds) | | | |

The composition according to the invention did not whiten after application to wet skin and n

It was also surprisingly found that the compositions according to the invention containing an acrylates/C10-30 alkyl acrylate crosspolymer, which is defined by the fact that, if the polymer is dissolved in a total quantity of 0.2% by weight in water and the resulting mixture is adjusted to a pH of 7.5 with NaOH, the resulting mixture has a viscosity in the range from 4500 mPa·s to 5500 mPa·s, whereby the viscosity is measured using a Brookfield RV viscometer, spindle RV 4, 20 rpm without Helipath, at 20° C. ambient temperature and 20° C. sample temperature, show in comparison to composition containing a different acrylates/C10-30 alkyl acrylate crosspolymer an improved stability, when being stored at 40° C., 1013 hPa and 50% humidity. The composition Inv. 1 was stable for at least 8 months.

The following examples shall further exemplify the present invention.

| INCI | Ex. 1 | Ex. 2 | Ex. 3 | Ex. 4 |
|---|---|---|---|---|
| Diisopropyl Adipate | 3 | 0 | 0 | 0 |
| Isodecyl Malate | 0 | 5 | 0 | 0 |
| Lauryl Lactate | 0 | 0 | 8 | 0 |
| C12-15 Alkyl Lactate | 0 | 0 | 0 | 10 |
| Dimethicone | 0.3 | 0.1 | 0.5 | 0.8 |
| Silica Dimethyl Silylate | 1 | 0.5 | 0.7 | 0.8 |
| Silica | 0.5 | 0.7 | 2 | 1.5 |
| Tapioca Starch | 0.85 | 1.7 | 1.3 | 2.4 |
| Fragrance | q.s. | q.s. | q.s. | q.s. |
| Propylene Glycol | 0 | 0.9 | 0.5 | 0.3 |
| Glycerin | 0.9 | 6 | 1 | 5 |
| Methylpropanediol | 2 | 2 | 2 | 2 |
| Sodium Hydroxide | q.s. | q.s. | q.s. | q.s. |
| Phenoxyethanol | 0.8 | 0.8 | 0.8 | 0.8 |
| Carbomer | 0 | 0.16 | 0 | 0.2 |
| Cetearyl Alcohol | 0 | 0 | 1 | 2 |
| Acrylates/C10-30 Alkyl Acrylate | 0.2 | 0.5 | 0.3 | 0.5 |
| Crosspolymer (Pemulen EZ4U, | | | | |
| Lubrizol) | | | | |
| Xanthan Gum | 0 | 0 | 0.4 | 0 |
| Alcohol Denat. | 3.8 | 2.9 | 5.8 | 4.8 |
| Trisodium EDTA | 0.2 | 0.18 | 0.14 | 0.16 |
| Homosalate | 8 | 8 | 8 | 9 |
| Octocrylene | 2 | 2 | 2 | 8 |
| Ethylhexyl Salicylate | 4.5 | 4.75 | 4.5 | 4.5 |
| Bis-Ethylhexyloxyphenol | 1 | 3.5 | 1 | 3.5 |
| Methoxyphenyl Triazine | | | | |
| Butyl Methoxydibenzoylmethane | 3.5 | 4.75 | 3.5 | 4.5 |
| Ethylhexyl Triazone | 0 | 4 | 0 | 0 |
| Phenylbenzimidazole Sulfonic | 0 | 2 | 1 | 1 |
| Acid | | | | |
| Polysilicone-15 | 0 | 0 | 2 | 0 |
| Aqua | ad 100 | ad 100 | ad 100 | ad 100 |

1.-19. (canceled)

20. A method of reducing the white appearance of a cosmetic composition after application thereof onto wet skin or of reducing the time until the white appearance of the composition disappears after the application to wet skin, wherein the method comprises using a cosmetic composition comprising
(a) at least one ester having a dielectric constant of at least 4.0 at a temperature of 25° C.;
(b) at least one acrylates/C10-30 alkyl acrylate crosspolymer which upon dissolution in water in a total concentration of 0.2% by weight and adjusting a resultant solution to a pH of 7.5 affords a viscosity of the solution of from 4500 mPa-s to 5500 mPa-s measured using a Brookfield RV viscometer, spindle RV 4, 20 rpm without Helipath, at 20° C. ambient temperature and 20° C. sample temperature; and
(c) at least 40% by weight of water, based on a total weight of the composition.

21. A cosmetic composition, wherein the composition comprises
(a) at least one ester having a dielectric constant of at least 4.0 at a temperature of 25° C.;
(b) at least one acrylates/C10-30 alkyl acrylate crosspolymer which upon dissolution in water in a total concentration of 0.2% by weight and adjusting a resultant solution to a pH of 7.5 affords a viscosity of the solution of from 4500 mPa-s to 5500 mPa-s, measured using a Brookfield RV viscometer, spindle RV 4, 20 rpm without Helipath, at 20° C. ambient temperature and 20° C. sample temperature; and)
at least 40% by weight of water, based on a total weight of the composition.

22. The composition of claim 21, wherein (a) is not an organic UV filter.

23. The composition of claim 21, wherein the dielectric constant of (a) is at least 4.5.

24. The composition of claim 21, wherein the constant dielectric of (a) is at least 5.2.

25. The composition of claim 21, wherein the dielectric constant of (a) is less than 8.0.

26. The composition of claim 21, wherein (a) consists of one or more of diisopropyl adipate, lauryl lactate, dibutyl adipate, diisopropyl sebacate, diethylhexyl adipate, isodecyl malate, and C₁₂₋₁₅ alkyl lactate.

27. The composition of claim 21, wherein is or comprises diisopropyl adipate.

28. The composition of claim 21, wherein the composition comprises from 1% to 12% by weight of (a), based on the total weight of the composition.

29. The composition of claim 21, wherein the composition comprises from 0.15% to 0.55% by weight of (b), based on the total weight of the composition.

30. The composition of claim 21, wherein a total concentration of acrylates/C10-30 alkyl acrylate crosspolymers which upon dissolution in water in a total concentration of 0.2% by weight and adjusting a resultant solution to a pH of 7.5 afford a viscosity of the solution of less than 4000 mPa-s and/or more than 6000 mPa-s, measured using a Brookfield RV viscometer, spindle RV 4, 20 rpm without Helipath, at 20° C. ambient temperature and 20° C. sample temperature, is 0% to less than 0.05% by weight, based on the total weight of the composition.

31. The composition of claim 21, wherein the total concentration of all non-silicone oils which have a dielectric constant of less than 4.0 at 25° C. and are not an organic UV filter is 0% to less than 1% by weight, based on the total weight of the composition.

32. The composition of claim 21, wherein a total concentration of C₁₂₋₁₅ alkyl benzoate, mineral oil, caprylic/capric triglyceride and/or ethylhexyl palmitate in the composition is 0% to less than 0.2% by weight, based on the total weight of the composition.

33. The composition of claim 21, wherein the composition further comprises from 0.1% to 1% by weight of one or more silicone oils, based on the total weight of the composition.

34. The composition of claim 21, wherein the composition further comprises one or more organic UV filters.

35. The composition of claim 34, wherein the composition comprises at least one of homosalate, phenylbenzimidazole sulfonic acid, butyl methoxydibenzoylmethane, octocrylene, ethylhexyl triazone, ethylhexyl salicylate, benzohenone-4, bis-ethylhexyloxyphenol methoxyphenyl triazine, and polysilicone-15.

36. The composition of claim 35, wherein the composition comprises at least one of bis-ethylhexyloxyphenol methoxyphenyl triazine, homosalate, ethylhexyl salicylate, octocrylene, and butyl methoxydibenzoylmethane in a total concentration of from 1% to 35% by weight, based on the total weight of the composition.

37. The composition of claim 21, wherein the composition further comprises from 1% to 10% by weight of ethanol, based on a total weight of the composition.

38. The composition of claim 21, wherein the composition comprises a gel structure.

39. The composition of claim 21, wherein the composition comprises 0% to less than 2% by weight of further emulsifiers, based on the total weight of the composition.
